(19) United States
(12) Patent Application Publication
(10) Pub. No.: US 2011/0139883 A1
(43) Pub. Date: Jun. 16, 2011

(54) VOLATILE MATERIAL DISPENSER AND METHOD OF RETAINING REFILLS IN SAME

(76) Inventors: Thomas P. Gasper, Germantown, WI (US); Chris A. Kubicek, East Troy, WI (US); Andrew N. Niemiec, Franklin, WI (US); Robert C. Coon, Chicago, IL (US); Bjorn Gunderson, Chicago, IL (US); Erik Joseph Moses, Carol Stream, IL (US); Mike O'Connor, Gurnee, IL (US)

(21) Appl. No.: 12/874,084

(22) Filed: Sep. 1, 2010

Related U.S. Application Data
(63) Continuation-in-part of application No. 12/761,846, filed on Apr. 16, 2010.
(60) Provisional application No. 61/286,575, filed on Dec. 15, 2009.

(51) Int. Cl. B05B 17/04; A61L 9/04

(52) U.S. Cl. 239/1; 239/44

(57) ABSTRACT

A volatile material dispenser includes a housing, a retention mechanism coupled to the housing, a first refill having a wick extending therefrom, and a first adapter removably attached to a portion of the first refill. The retention mechanism non-removably retains the first adapter to the housing when the first refill and the first adapter combination are inserted therein. When the first refill is removed from the dispenser, the first adapter remains in the dispenser such that a second refill with a second adapter removably attached thereto can be inserted into and retained by the first adapter.

### CROSS REFERENCE TO RELATED APPLICATIONS

The present application is a continuation-in-part of U.S. patent application Ser. No. 12/761,846, filed on Apr. 16, 2010, and entitled “Volatile Material Dispensers,” which claims the benefit of U.S. Provisional Application Ser. No. 61/286,575, the entireties of which are incorporated herein by reference.

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

The present invention relates generally to volatile material dispensers and, more particularly, to volatile material dispensers that are adapted to be functionally coupled with only specific volatile material refills.

2. Description of the Background of the Invention

Multiple different volatile material dispensers are commercially sold and generally include a housing and a volatile material refill that is inserted into the housing. The refill generally includes a container or bottle for holding a volatile material therein. In some dispensers, the volatile material is passively emitted therefrom. In other dispensers, a diffusion element is utilized to facilitate the dispensing of the volatile material. Examples of diffusion elements include heaters, piezoelectric elements, fans, aerosol actuators, and the like. Regardless of the manner in which the volatile material is emitted, once the volatile material has been expended from the refill, the refill can typically be removed by a user and replaced with a new refill.

One type of commercial volatile material dispenser, referred to herein as a plug-in scented oil dispenser, includes a housing and a heater disposed within the housing. A refill for use with a plug-in scented oil dispenser generally includes a container portion having a bottom end and a top end, wherein the container portion terminates in a neck portion at the top end. A volatile material is disposed within the container portion and a wick is in contact with the volatile material and extends out of the refill through the neck portion. A plug or other connector generally positions and retains the wick within the neck portion. Upon insertion of the refill into the dispenser, at least a portion of the wick is disposed adjacent the heater such that volatile material that moves through the wick is volatilized by the heater.

Another feature of various volatile material dispensers and refills is that each refill has features that are unique or complementary to the particular dimensions of the housing of the dispenser for which it is sold. Still further, each type of dispenser is generally adapted to accept only a single type of refill having features unique or complementary to that dispenser. For example, plug-in scented oil dispensers sold by S.C. Johnson & Son, Inc. (“S.C. Johnson”) of Racine, Wis., generally only accept refills sold by S.C. Johnson under their GLADE® brand and such refills are designed to fit only within S.C. Johnson plug-in scented oil dispensers. Similarly, plug-in scented oil dispensers sold by Reckitt Benckiser (“Reckitt”) of Berkshire, England generally only accept refills sold by Reckitt under their AIR WICK® brand and such refills are designed to generally fit only within Reckitt plug-in scented oil dispensers. Still similarly, refills sold for use with plug-in scented oil dispensers sold by Procter & Gamble (“P&G”) of Cincinnati, Ohio are generally designed to fit only with P&G plug-in scented oil dispensers. This is generally the case for most commercial plug-in scented oil dispensers and refills therefor.

### SUMMARY OF THE INVENTION

According to one embodiment, a method of retaining refills in a volatile material dispenser includes the step of providing a first refill having a container with a volatile material therein, a neck portion extending upwardly from the container, and a wick in contact with the volatile material and extending out of the refill through the neck portion. The method further includes the step of providing a first adapter removably attached to the neck portion of the first refill. Still further, the method includes the step of providing instructions for insertion of the first refill with the first adapter attached thereto into the volatile material dispenser so that a retention mechanism within the volatile material dispenser fixedly retains the first adapter and the first refill to the housing. The method further includes the step of providing instructions for removal of the first refill from the first adapter, whereby upon removal, the first adapter is retained within the dispenser. Still further, the method includes the step of providing instructions for insertion of a second refill having a second adapter removably attached to a second neck portion thereof into the volatile material dispenser such that the second refill is retained in the dispenser.

According to another embodiment, a volatile material dispenser includes a housing, a retention mechanism coupled to the housing, a first refill having a wick extending therefrom, and a first adapter removably attached to a portion of the first refill. The retention mechanism non-removably retains the first adapter to the housing when the first refill and the first adapter combination are inserted therein. When the first refill is removed from the dispenser, the first adapter remains in the dispenser such that a second refill with a second adapter removably attached thereto can be inserted into and retained by the first adapter.

According to yet another embodiment, a method of retaining refills in a volatile material dispenser includes the step of inserting a first refill having a container with a volatile material therein, a neck portion extending upwardly from the container, a wick in contact with the volatile material and extending out of the refill through the neck portion, and a first adapter removably attached to the neck portion into a volatile material dispenser having a retention mechanism so that the retention mechanism fixedly retains the first adapter and the first refill to the housing. The method further includes the step of removing the first refill from the dispenser, whereby upon removal, the first adapter is retained within the dispenser. Still further, the method includes the step of inserting a second refill having a second adapter removably attached to a second neck portion thereof into the volatile material dispenser such that the second refill is retained in the dispenser.

### BRIEF DESCRIPTION OF THE DRAWINGS

FIG. 1 is a top isometric view of a volatile material dispenser with a compatible refill retained therein;

FIG. 2 is a cross-sectional view taken generally along the lines 2-2 of FIG. 1 with portions of a housing of the dispenser removed therefrom;

FIG. 3 is a front elevational view of a compatible refill in combination with a first embodiment of an adapter, wherein the combination is retained by a retention mechanism and the adapter and portions of the dispenser are shown in cross-section;

FIG. 4 is a front elevational view of a compatible refill in combination with a second embodiment of an adapter, wherein the combination is retained by a retention mechanism and the adapter and portions of the dispenser are shown in cross-section;

FIG. 5 is a top isometric view of a compatible refill;

FIG. 6 is a top isometric view of the compatible refill of FIG. 5 with a further embodiment of an adapter attached thereto;

FIG. 7 is a cross-sectional view taken generally along the lines 7-7 of FIG. 6;

FIG. 8 is a cross-sectional view taken generally along the lines 2-2 of FIG. 1 with the refill and adapter of FIG. 6 inserted into the dispenser of FIG. 1;

FIG. 9 is a cross-sectional view similar to that of FIG. 8 with the refill removed therefrom, leaving behind the adapter;

FIG. 10 is a cross-sectional view similar to that of FIG. 8 with a further refill inserted into the dispenser and retained therein by the adapter of FIG. 9; and

FIG. 11 is a cross-sectional view similar to that of FIG. 8 depicting an inability to insert an incompatible refill into the dispenser due to the presence of the adapter of FIG. 9 therein.

Other aspects and advantages of the present invention will become apparent upon consideration of the following detailed description, wherein similar structures have like or similar reference numerals.

### DETAILED DESCRIPTION

The present invention is directed to volatile material dispensers for vaporizing and dispensing volatile materials and methods of retaining refills in same. While the present invention may be embodied in many different forms, several specific embodiments are discussed herein with the understanding that the present invention is to be considered only as an exemplification of the principles of the invention, and it is not intended to limit the invention to the embodiments illustrated.

Further, the use of the term volatile material herein refers to any volatile material that a consumer may desire to emit into an area surrounding one or more refills holding the volatile material(s) and/or a dispenser holding one or more refills. Illustratively, the types of volatile materials may be, for example, a cleaner, an insecticide, an insect repellant, an insect attractant, a mold or mildew inhibitor, a fragrance, a disinfectant, an air purifier, an aromatherapy scent, an antiseptic, a positive fragrancing volatile material, an air-freshener, a deodorizer, or the like, and combinations thereof. Additives may be included in the volatile material, such as, for example, fragrances and/or preservatives.

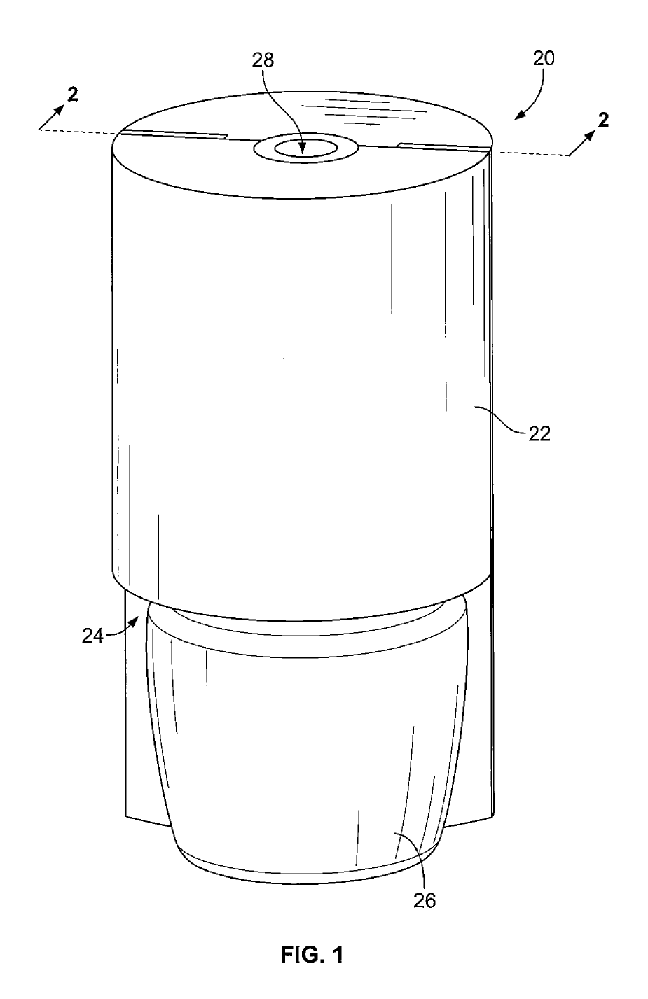

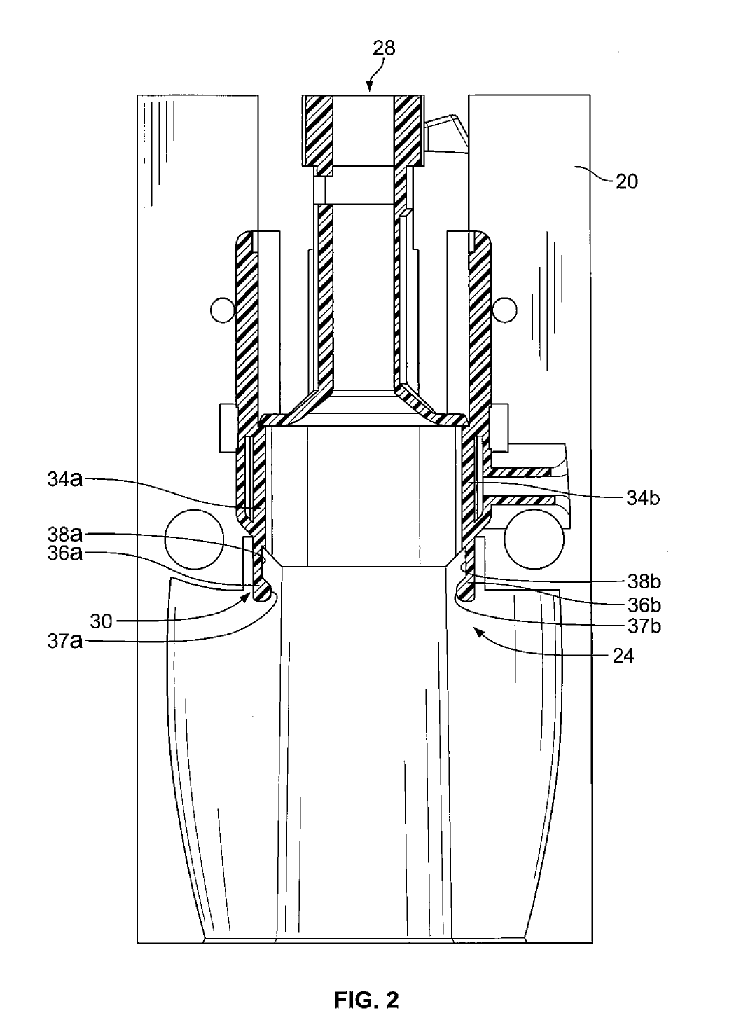

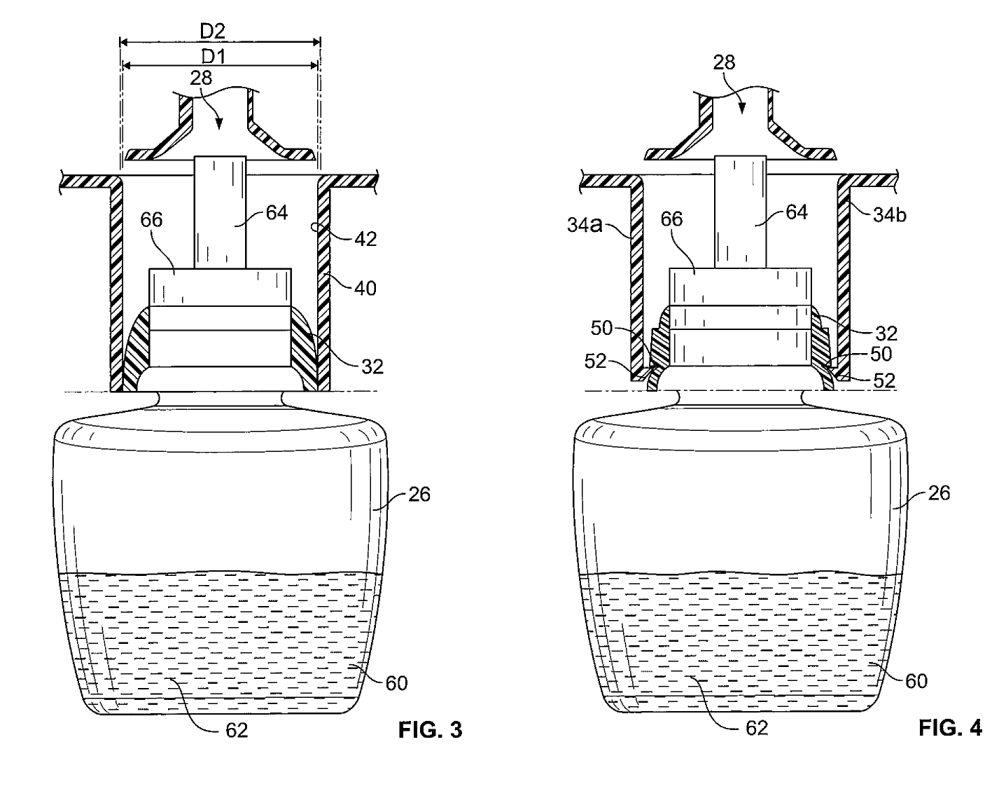

Referring to FIGS. 1 and 2, a volatile material dispenser 20 includes a housing 22 having a cavity 24 for accepting a compatible volatile material refill 26 and an optional diffusion element or elements (not shown) disposed in the housing 22. The diffusion element may be a heater, a fan, a piezoelectric element, or any other diffusion element or combination of diffusion elements known in the art. The housing 22 further includes a channel 28 for receiving a wick disposed within and extending from the refill 26 and a retention mechanism 30 for attaching the refill 26 to the housing 22. Alternatively or in conjunction, the retention mechanism 30 is configured to retain an adapter 32 (for example, as seen in FIGS. 3, 4, and 6) attached to the refill 26 to the housing 22, as will be discussed in greater detail hereinafter. The retention mechanism 30, as seen in FIG. 2, includes walls 34a, 34b extending downwardly within the cavity 24 of the dispenser 20. Each of the walls 34a, 34b terminates in a latch 36a, 36b having an outwardly extending projection 37a, 37b and may include a groove 38a, 38b formed adjacent the latch 36a, 36b. The latches 36a, 36b and/or grooves 38a, 38b, in one embodiment, are configured to fixedly retain the adapter 32 of the refill 26. Optionally, the latches 36a, 36b alone may retain the adapter 32, and thus the refill 26.

The retention mechanism 30 may alternatively, or in addition, include a cylindrical wall 40 depending into the cavity 24, as seen in FIG. 3 and discussed in greater detail hereinafter, and/or any other structure that would retain the adapter 32 and refill 26. In one embodiment (FIG. 3), the wall 40 is rigid and does not include a structure to retain the adapter 32, but instead has a diameter D1 formed by an inner surface 42 of the wall 40 that is smaller than a largest diameter D2 of the adapter 32, thereby forming a friction fit therebetween. Preferably, the fit between the retention mechanism 30 and the adapter 32 is one that either prevents removal of or makes removal of the adapter 32 very difficult. Examples of such other fits include adhesives, an interference fit, a friction fit, a snap fit, and the like.

The retention mechanism 30 may alternatively, or in addition, include any other structure(s) that would retain of the adapter 32 and refill 26 within the dispenser. In one embodiment, the adapter 32 includes one or more grooves 50 disposed therein and the retention mechanism 30 includes one or more flexible projections 52 that interact with the grooves 50 to retain the adapter 32, as seen in FIG. 4. Alternatively, the adapter 32 may include one or more projections and the retention mechanism 30 may include one or more grooves. The retention mechanism 30 may take any form, as long as it retains the adapter 32 and refill 26 therein and prevents removal of the adapter 32 upon removal of the refill 26.

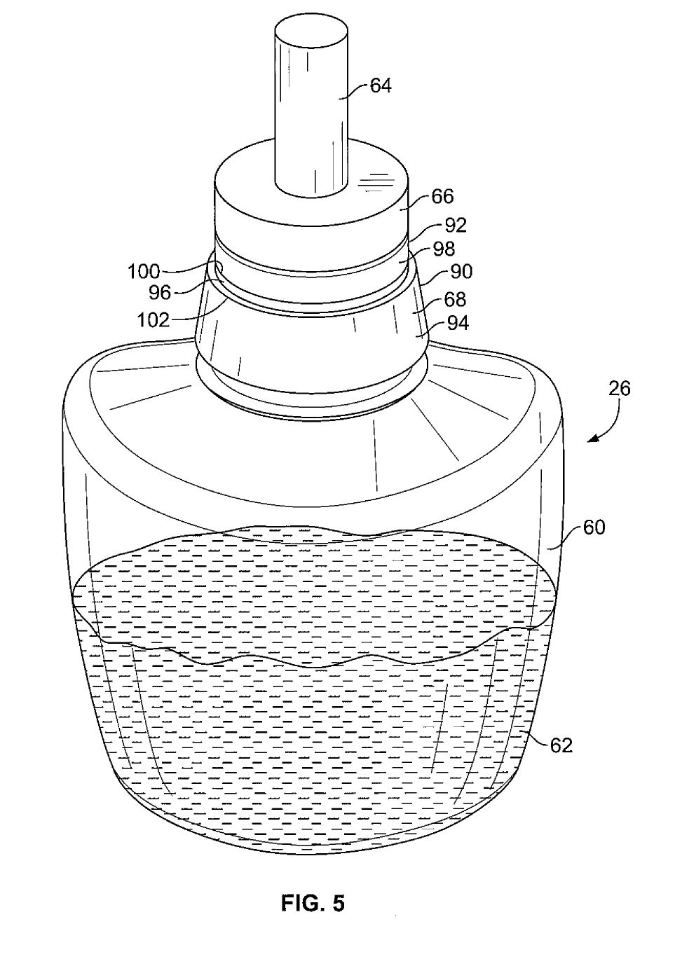

As best seen in FIG. 5, the compatible refill 26 includes a bottle or container 60 with a volatile material 62 disposed therein. The volatile material 62 disposed in the container 60 may be of any type of volatile material adapted to be dispensed into the air. An elongate wick 64 is in contact with the volatile material 62 in the container 60 and extends from the container 60 so that at least a portion of the wick 64 is exposed to the ambient environment. A plug assembly 66 holds the wick 64 in place with respect to the container 60. The wick 64 is depicted as a substantially uniform cylinder with a substantially constant diameter throughout an entire length thereof. However, in other embodiments, the wick 64 may be other shapes and sizes as known in the art. The wick 64 may be made of one material or alternatively may be made of multiple materials, e.g., polymeric or porous materials and/or any other material known in the art that are suitable for wicking.

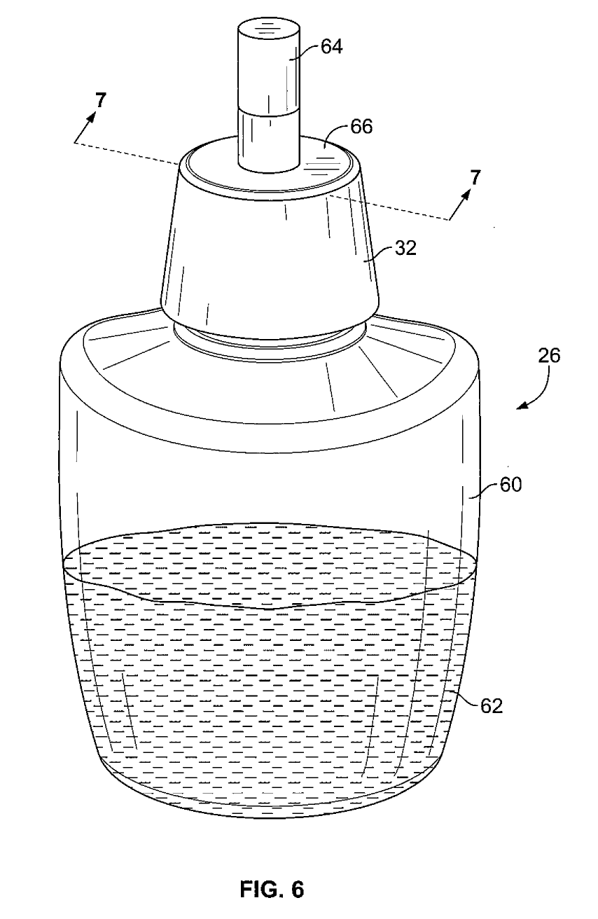

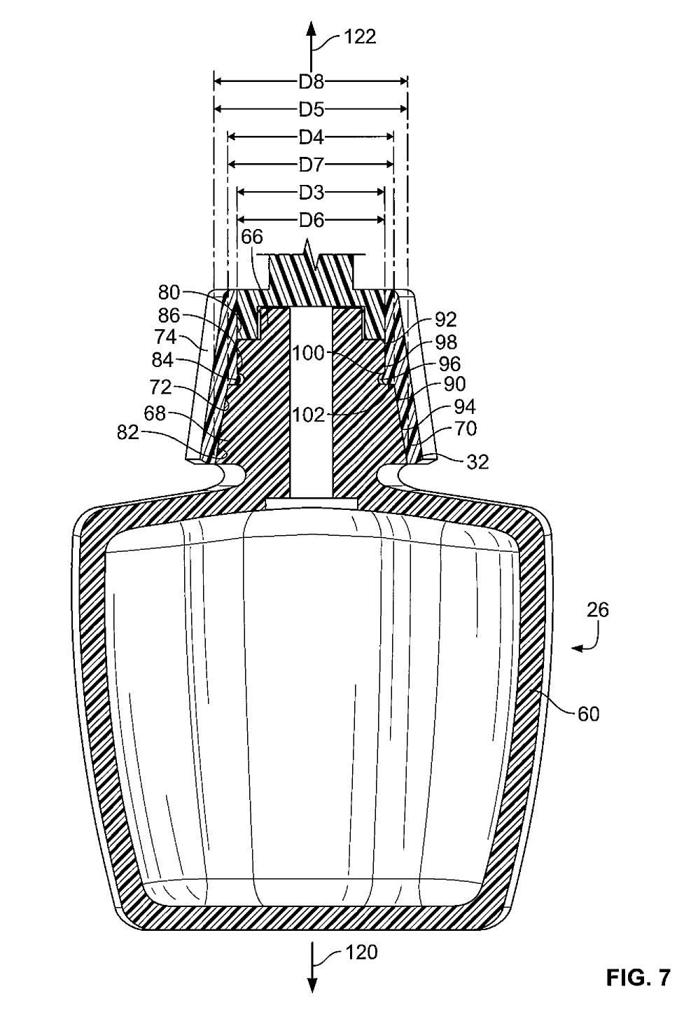

A further embodiment of the adapter 32, as seen in FIGS. 6 and 7, is removably attached to a neck portion 68 (FIG. 5) that extends upwardly from the container 60. Referring to FIG. 7, the adapter 32 includes a generally frustro-conical wall 70 with an inner surface 72 and an outer surface 74, wherein the wall 70 and the surfaces 72, 74 are generally angled or tapered outwardly from top to bottom. The wall 70 and the surfaces 72, 74 are preferably disposed at an angle greater than 0 with respect to a longitudinal axis of the container 60. The inner surface 72 has a first, upper portion 80 and a second, lower portion 82 joined at an inwardly extending annular projection 84. The upper portion 80 is generally vertical, has a diameter D3, and ends at a lower end 86 thereof in the projection 84. The lower portion 82 begins at the projection 84 with a diameter D4 that is greater than the diameter D3 and tapers outwardly to a diameter D5, which is greater than the diameter D4.

Referring to FIGS. 5 and 7, the neck portion 68 of the refill 26 includes structure complementary to that of the inner surface 72 of the adapter 32. In particular, the neck portion 68 of the refill 26 has an outer surface 90 that is angled or tapered at approximately the same angles as the inner surface 72 of the adapter 32. The outer surface 90 includes a first, upper portion 92 and a second, lower portion 94 connected by a groove 96. The upper portion 92 of the neck portion 68 is generally vertical, has a diameter D6 that is similar to the diameter D3, and ends at a lower end 98 thereof in the groove 96. The lower portion 94 begins at the groove 96 with a diameter D7 that is similar to the diameter D4 and greater than the diameter D6. The lower portion 94 tapers outwardly to a diameter D8, which is similar to the diameter D5 and greater than the diameter D7.

The similar size and shape of the inner surface 72 of the adapter 32 and the outer surface 90 of the neck portion 68 allow the adapter 32 to be retained on the refill 26, but also allow the refill 26 to be removed from the adapter 26 in a downward direction 120 (FIG. 7). In addition, the groove 96 is shallower at an upper edge 100 thereof than at a lower edge 102 thereof to allow the adapter 32 to be pulled in the direction 120, but prevent movement of the adapter 32 in an opposite direction 122. In use within a dispenser, this allows the refill 26 to be removed from the dispenser without the adapter 32, thereby leaving the adapter 32 behind within the dispenser, as discussed in greater detail hereinafter.

Although the projection 84 and groove 96 are depicted as continuous, in other embodiments, the projection 84 and/or groove 96 may be discontinuous and/or may include a plurality of projections and/or grooves. Still further, any other attachment mechanism known in the art may be utilized to retain the adapter 32 on the refill 26, so long as the refill 26 may be removed from the adapter 32 as the container 40 is pulled away from the adapter 32 (to allow the adapter 32 to remain within the dispenser 20). Such attachment mechanisms include a threaded engagement, a bayonet-type coupling, an interference fit, a friction fit, or any other means that would be apparent to one skilled in the art. In other embodiments, the adapter 32 can be attached to other portions of the container without departing from the spirit of the present invention.

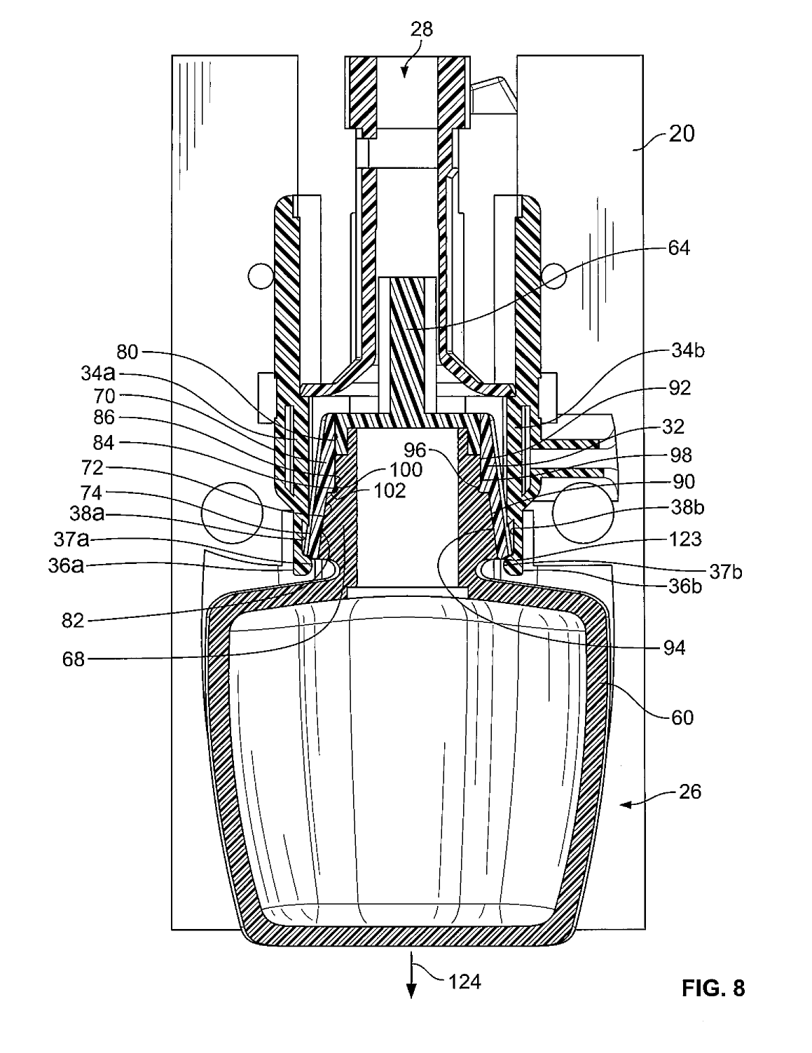

FIG. 8 depicts the refill 26 with adapter 32 attached thereto inserted into and retained within the dispenser 20. The wick 64 extending from the refill 26 is inserted into the channel 28 and, once the wick 64 is aligned within the channel 28, the refill 26 is moved upwardly into the dispenser 20 and the latches 36a, 36b move outwardly to accommodate the adapter 32. As the adapter 32 passes the latches 36a, 36b, the latches 36a, 36b return to their original position and interfere with a lowermost edge 123 of the adapter 32 such that the adapter 32 is retained by the latches 36a, 36b. If grooves 38a, 38b are present in the walls 34a, 34b, all or a portion of the adapter 32 may reside within and be retained by the grooves 38a, 38b. Whether or not the grooves 38a, 38b are utilized, the adapter 32 may additionally or alternatively be retained by a friction fit between the adapter 32 and the walls 34a, 34b, as discussed hereinabove. In one embodiment, the adapter 32 is non-removably or fixedly attached to the housing 22 of the dispenser 20 by the latches 36a, 36b and/or the grooves 38a, 38b.

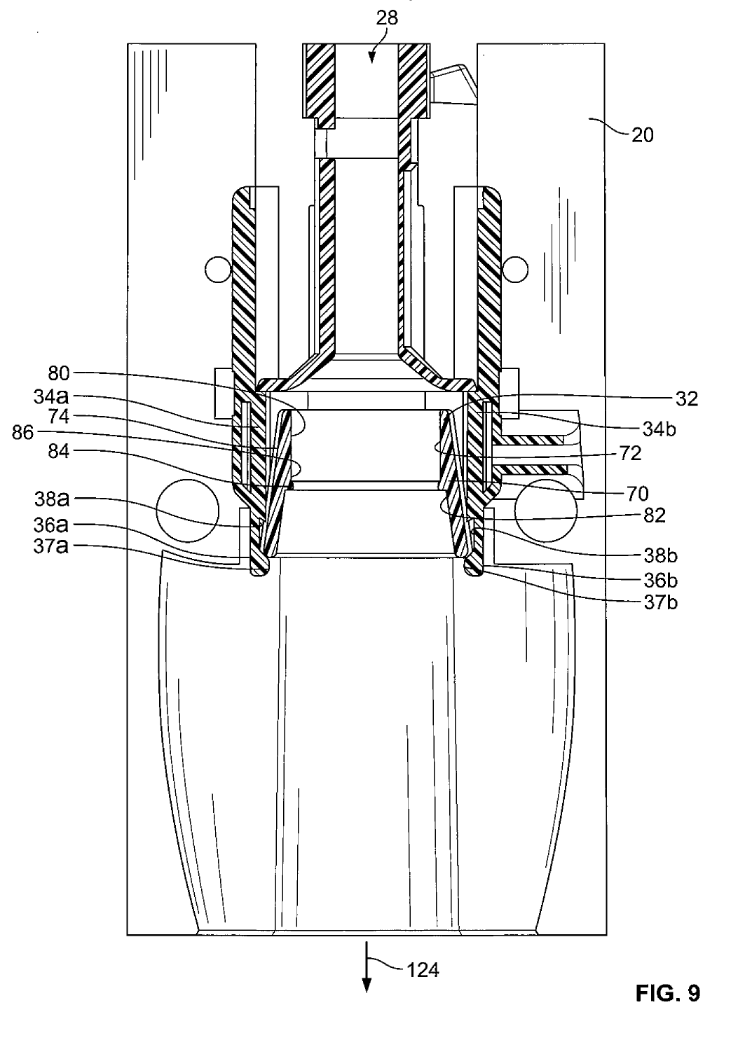

After the refill 26 and the adapter 32 combination are attached to the dispenser 20, at least a portion of the wick 64 is preferably exposed to allow a diffusion element, preferably in the form of a heater, to act upon the wick 64 to evaporate and dispense the volatile material 62 that is moved to the exposed portion of the wick 64 through capillary action. Once the volatile material 62 within the container 60 becomes depleted (or before such point, if desired), a user can replace the refill 26 with a further refill that contains volatile material. In the present embodiment, the refill 26 may be detached from the adapter 32 so that the adapter 32 remains attached to the housing 22, as shown in FIG. 9. In particular, the shape and dimensions of the inner surface 72 of the adapter 32 and the outer surface 90 of the neck portion 68 of the refill 26 allow the refill 26 to be pulled downwardly in the direction 124, which allows the refill 26 to be removed, leaving the adapter 32 behind in the dispenser 20. In various examples, the refill 26 may be detached from the adapter 32 by unscrewing the refill 26, exerting a downward force on the refill 26, or otherwise decoupling the refill 26 in any other known method, depending on the type of attachment mechanism utilized.

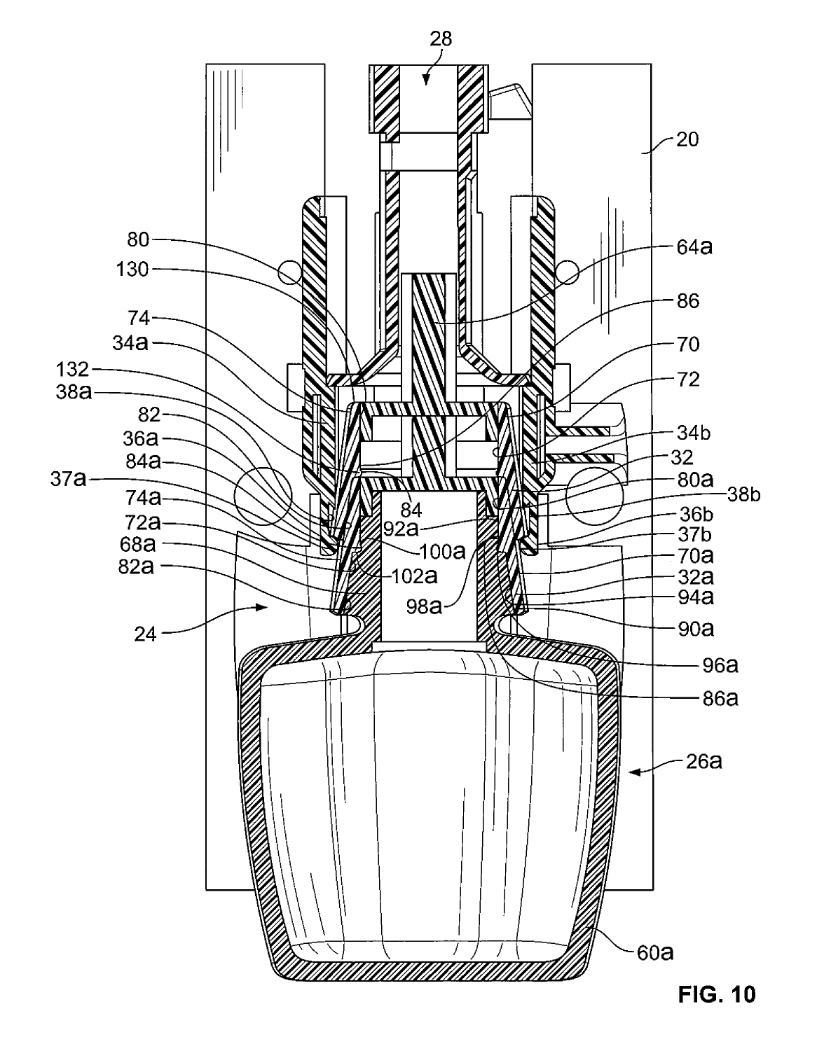

Subsequently, as shown in FIG. 10, a further compatible refill 26a can be inserted into and retained by the adapter 32. In particular, the further refill 26a is inserted into the dispenser 20 in a similar manner as described above. As the further refill 26a is inserted upwardly, a further adapter 32a of the further refill 26a contacts the original adapter 32 and, the adapter 32a is wedged or nested into the adapter 32 and retained therein by a friction fit. Specifically, an upper edge 130 of the adapter 32a abuts a lower edge 132 of the projection 84 of the original adapter 32 and the outer surface 70a of the further refill 26a is wedged or nested against the lower portion 82 of the inner surface 72 of adapter 32 to create a friction fit. Parts of the refill 26a common to the refill 26 are labeled with the same reference numerals and the suffix “a”. The adapters 32, 32a are preferably made of a material, such as rubber, flexible plastic, and the like, that allows a friction fit, however any material type (plastic, metals, glass) could be designed to work. When a user desires to replace the further refill 26a, the further refill 26a and the further adapter 32a (but not the original adapter 32) are removed and yet another refill and adapter are inserted therein. In further embodiments, the adapters 32, 32a include other features that allow similar adapters to be detachably attached to one another, but prevent incompatible refills from being attached to the adapter 32, and thus the dispenser 20. For example, the adapters may be attached in any manner known in the art including, for example, a snap fit, threading, bayonet-type connection, an interference fit, and/or the like, but preferably in a manner that prevents attachment of other refills.

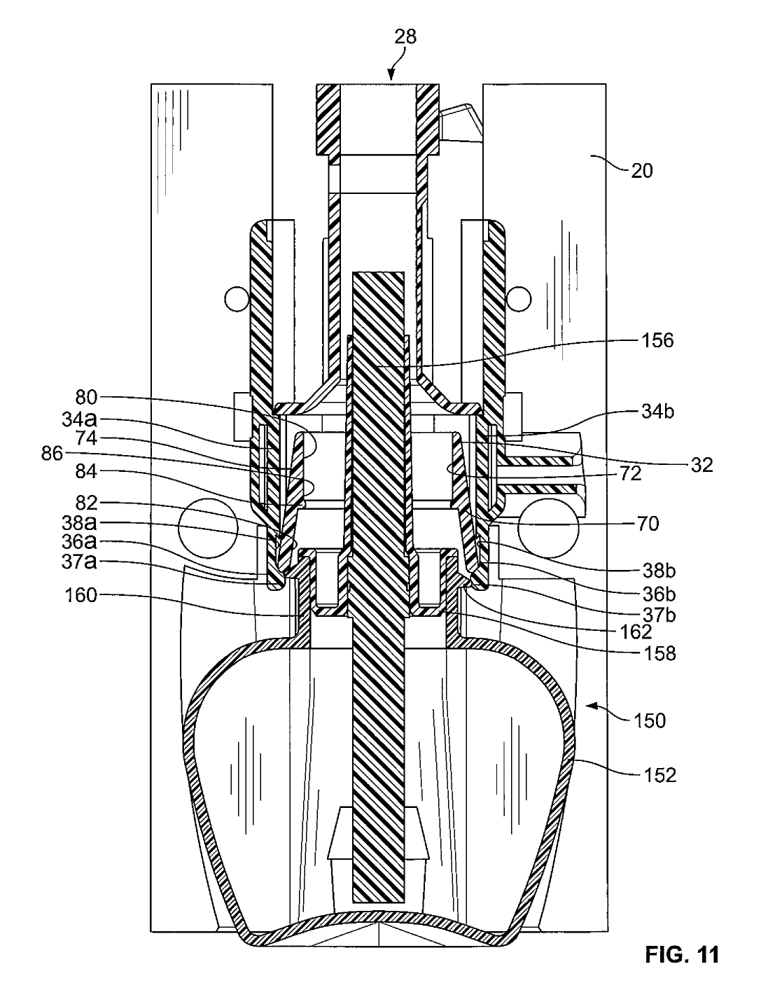

Once the adapter 32 remains in the dispenser 20 and cannot be easily removed, the adapter 32 prevents insertion and retention of an incompatible refill 150 because the latches 36a, 36b thereof are no longer accessible to retain a refill therein. An incompatible refill 150 is a refill having a different configuration, coupling feature(s), and/or dimensions that prevent the incompatible refill from being inserted into and retained by the dispenser 20 once the adapter 32 is disposed therein. In various contemplated embodiments, the adapter 32 can prevent an incompatible refill from being attached to the housing 22 if, for example, the incompatible refill has a neck portion 68 that is too large or too small, has reverse threading, does not include coupling features complementary to the adapter 32, or any other known means. In one embodiment, the incompatible refill 150 would otherwise be capable of being attached to the housing 22 if not for the adapter 32 being disposed within the housing 22. For example, the incompatible refill 150 may include a retention structure (see threading in FIG. 11), for example, threading, one or more projections or grooves, and the like that interferes with the latches 36a, 36b to attach and retain the incompatible refill 150 to the housing 22. In the present embodiment, the coupling of the adapter 32 to the housing 22 prevents the latches 36a, 36b from retaining the retention structure or any other portion of the incompatible refill 150, and the adapter 32 prevents attachment of the refill 150, thereby rendering the incompatible refill 150 useless with the dispenser 20.

As shown in FIG. 11, one example of an incompatible refill 150 includes a bottle or container 152 with a volatile material (not shown) disposed therein. An elongate wick 156 is in contact with the volatile material and extends from the container 152 so that at least a portion of the wick 156 is exposed. A plug assembly 158 holds the wick 156 within a neck portion 160 of the container 152. The neck portion 160 includes threading 162 thereon for attachment of a cap (not shown) and/or attachment to a dispenser. As noted above, the incompatible refill 150 is incapable of being inserted into the dispenser 20 once the adapter 32 resides therein because the size of the neck portion 160 of the refill 150 does not correspond to a size of the adapter 32, nor does the neck portion 160 have features that are complementary to those of the adapter 32. If the adapter 32 were not present within the dispenser 20 of FIG. 11, the refill 150 would be retained within the dispenser 20 by an interaction between the latches 36a, 36b and the threading 162 on the neck portion 160 of the refill 150.

Although the dispensers described herein are disclosed as retaining a compatible or first refill with an adapter attached thereto and, upon removal of the compatible refill and retention of the adapter within the dispenser, prevent insertion of incompatible or differently configured refills, the present invention is not limited by such disclosure. In fact, the incompatible or second refill could include the adapter, wherein upon insertion and removal of the incompatible refill, the adapter is retained within the dispenser to prevent insertion of the compatible refill. In such case, the incompatible refill could be capable of being retained within the dispenser without the adapter or could optionally have dimensions that do not allow the incompatible refill to be retained within the dispenser without the adapter.

Other embodiments of the adapter 32 are contemplated, wherein the adapter 32 may be attached to any portion of the container 60, the neck portion 68, the plug assembly 66, and/or the wick 64. The relevant features of the adapter 32 are a mechanism for attachment to the refill 32 and a further mechanism for permanent or semi-permanent attachment to the housing 22 (via the latches 36a, 36b). Various embodiments of the adapter 32 may be attached to a refill 26 in any manner known in the art including, for example, a snap fit, threading, bayonet-type connection, an interference fit, and/or the like, as long as such attachment allows for removal of the refill 26 from the adapter 26, such that the adapter 16 remains in the dispenser 20.

Although the adapters 32 herein are attached to the housing 22 of the dispenser 20 after insertion and removal of a first refill 26, in other embodiments, the adapter 32 alone may be attached to the housing 22 during manufacture of the dispenser 20 or a user may be directed to attach the adapter 32 to the housing 22 upon first use of the dispenser 20. In any of the embodiments disclosed herein, instructions may be provided with the dispenser 20 and/or refill as to how to insert and removal refills into the dispenser 20. Such instructions may be in the form of advertising on the packaging thereof, a booklet contained within the packaging thereof, and/or any other manner that is known to convey to a consumer how to properly use the refill with the dispenser 20.

The dispenser employing the refills with adapters described herein may comprise a variety of forms as known in the art. The dispenser may include any number of electrical or mechanical features that aid in vaporizing and/or dispensing the volatile material, provide aesthetics for the dispenser, and/or provide other functional features to the dispenser, as long as features are present in the dispenser to retain the adapter and refill therein. For example, the dispenser may include one or more of a heater, a fan, and/or other diffusion elements disposed in a housing that help facilitate the release of the volatile active.

The dispenser may optionally use electric power in the form of batteries or an electrical plug during the operation thereof. The dispenser may further include one or more openings in the housing to allow the volatile material to be dispensed from the housing 22 to the surrounding environment. In place of the latches 36a, 36b, the housing 22 may include a variety of internal implements or couplings to help secure the compatible refills with adapters disclosed herein, such as, for example, snaps, ridges, undercuts, lips, notches, projections, grooves, interference fit, threading, and/or any other attachment methods known in the art. The dispenser may optionally accommodate one or more refills and may operate using a variety of timing sequences as known in the art.

Although directional language, such as top, bottom, upper, lower, etc., is used herein, such terminology is meant to describe the relation of components herein and not to limit the scope of the present invention. In particular, such directional language is utilized in the case that the dispenser 20 and refill 26 are disposed in a use position and, as one skilled in the art would recognize, the dispenser 20 and/or refill 26 may be reoriented, but the same relative nature of the components applies.

Any of the embodiments described herein may be modified to include any of the structures or methodologies disclosed in connection with other embodiments.

### INDUSTRIAL APPLICABILITY

The present invention provides a volatile material dispenser that is adapted for use with only compatible volatile material refills. In particular, a compatible refill with an adapter that can be removably detached from the refill is inserted into the dispenser and retained thereby. The refill may be removed such that the adapter remains and the adapter remaining in the dispenser prevents incompatible refills from being coupled or attached to the dispenser, but allows retention of compatible refills having adapters attached thereto.

Numerous modifications to the present disclosure will be apparent to those skilled in the art in view of the foregoing description. Accordingly, this description is to be construed as illustrative only and is presented to enable those skilled in the art to make and use the disclosure and to teach the best mode of carrying out same. The exclusive rights to all modifications that come within the scope of the appended claims are reserved.

We claim:

1. A method of retaining refills in a volatile material dispenser, the method comprising the steps of:
providing a first refill having a container with a volatile material therein, a neck portion extending upwardly from the container, and a wick in contact with the volatile material and extending out of the refill through the neck portion;
providing a first adapter removably attached to the neck portion of the first refill;
providing instructions for insertion of the first refill with the first adapter attached thereto into the volatile material dispenser so that a retention mechanism within the dispenser fixedly retains the first adapter and the first refill to the housing;
providing instructions for removal of the first refill from the first adapter, whereby upon removal, the first adapter is retained within the dispenser; and
providing instructions for insertion of a second refill having a second adapter removably attached to a second neck portion thereof into the volatile material dispenser such that the second refill is retained in the dispenser.

2. The method of claim 1 further including the step of preventing removal of the first adapter from the dispenser and thereby preventing use of the retention mechanism.

3. The method of claim 1 further including the step of retaining the second refill nested within the first adapter.

4. The method of claim 3 further including the steps of providing the first and second adapters with inner and outer surfaces and inserting the second refill into the dispenser such that a friction fit is created between an outer surface of the second adapter and an inner surface of the first adapter.

5. The method of claim 4 further including the steps of providing instructions for removal of the second refill from the first adapter with the second adapter still attached to the second refill and providing instructions for attachment of a further refill for to the first adapter.

6. The method of claim 4 further including the step of providing attachment mechanisms on the inner surfaces of the first and second adapters that allow the adapters to be removed from respective refills in a first direction, but not in a second direction.

7. The method of claim 6, wherein each attachment mechanism includes a projection disposed on the inner surface of the respective adapter and a groove disposed in the neck portion of the respective refill such that the projection is retained in the groove to retain the adapter on the refill.

8. The method of claim 7 further including the steps of tapering the inner and outer walls from upper to lower ends thereof and tapering the neck portion of the refill in the same manner such that movement of the container away from the adapter is facilitated by the tapering and movement toward the adapter is prevented by the tapering.

9. The method of claim 1, further including the step of providing a volatile material dispenser.

10. The method of claim 1, wherein the instructions are provided in the form of advertising.

11. A volatile material dispenser, comprising:
a housing;
a retention mechanism coupled to the housing;
a first refill having a wick extending therefrom;
a first adapter removably attached to a portion of the first refill,
wherein the retention mechanism non-removably retains the first adapter to the housing when the first refill and first adapter combination are inserted therein; and
wherein when the first refill is removed from the dispenser, the first adapter remains in the dispenser such that a second refill with a second adapter removably attached thereto can be inserted into and retained by the first adapter.

12. The volatile material dispenser of claim 11, wherein the first and second adapters are sized and dimensioned such that an upper end of the second adapter fits within a lower end of the first adapter.

13. The volatile material dispenser of claim 12, wherein the second adapter fits securely within the first adapter by a friction fit created by an outer surface of the second adapter and an inner surface of the first adapter.

14. The volatile material dispenser of claim 13, wherein each of the first and second adapters includes an attachment mechanism on an inner surface thereof that allows the adapters to be removed from respective refills in a first direction, but not a second direction and wherein each attachment mechanism includes a projection disposed on the inner surface of the respective adapter and a groove disposed in the neck portion of the respective refill such that the projection is retained in the groove to retain the adapter on the refill.

15. The volatile material dispenser of claim 11, wherein the first and second adapters are removable from the first and second refills, respectively, in a first direction, but not in a second direction.

16. A method of retaining refills in a volatile material dispenser, the method comprising the steps of:
inserting a first refill having a container with a volatile material therein, a neck portion extending upwardly from the container, a wick in contact with the volatile material and extending out of the refill through the neck portion, and a first adapter removably attached to the neck portion into a volatile material dispenser having a retention mechanism so that the retention mechanism fixedly retains the first adapter and the first refill to the housing;
removing the first refill from the dispenser, whereby upon removal, the first adapter is retained within the dispenser; and
inserting a second refill having a second adapter removably attached to a second neck portion thereof into the volatile material dispenser such that the second refill is retained in the dispenser.

17. The method of claim 16, wherein the second refill is nested within the first adapter to retain the second refill in the dispenser.

18. The method of claim 17, wherein the first and second adapters include tapered inner and outer surfaces and a friction fit is created between the outer surface of the second adapter and the inner surface of the first adapter.

19. The method of claim 16, further including the step of removing the second refill from the dispenser with the second adapter still disposed thereon.

20. The method of claim 19, further including the step of inserting a further refill having a further adapter removably attached to a further neck portion thereof into the dispenser for retention of the further adapter within the first adapter.
